(19) United States
(12) Patent Application Publication
(10) Pub. No.: US 2020/0330286 A1
(43) Pub. Date: Oct. 22, 2020

(54) APPARATUS AND METHOD FOR FORMING AN ABSORBENT PAD

(71) Applicant: GDM S.p.A., Bologna (IT)

(72) Inventors: Marco ROSANI, Vailarate (Cremona) (IT); Matteo PIANTONI, Albino Fr. Fiobbio (Bergamo) (IT); Massimo RAGUSA, Monte Cremasco (Cremona) (IT); Giuseppe POLI, Cumignano sul Naviglio (Cremona) (IT); Federico TOSCANI, Castelleone (Cremona) (IT)

(21) Appl. No.: 16/760,321

(22) PCT Filed: Oct. 26, 2018
(86) PCT No.: PCT/IB2018/058368

§ 371 Date: Apr. 29, 2020

(30) Foreign Application Priority Data
Oct. 30, 2017 (IT) 102017000123038

(51) Int. Cl. A61F 13/15; A61F 13/532

(52) U.S. Cl. CPC A61F 13/15658; A61F 13/532; A61F 2013/15869; A61F 13/15764; A61F 2013/15991; A61F 13/15707

(57) ABSTRACT

An apparatus for forming an absorbent pad for an absorbent sanitary article including a first layer, a second layer and an absorbent material interposed between the first and the second layer, includes: a forming drum; a first feed system for feeding to the forming drum a first web, intended to form the first layer; and a second feed system for feeding to the forming drum a second web, intended to form the second layer; at least one spreader for spreading the absorbent material on the forming drum; the first web, the second web and the absorbent material together form a composite web and the forming apparatus includes a system for joining the first and second webs and a system for calibrating the thickness of the absorbent material in the composite web and being located upstream of the joining system in a feed direction V of the composite web.

### TECHNICAL FIELD

This invention relates to an apparatus and a method for forming an absorbent structure, specifically an absorbent pad or “core”, as it also known in the trade, intended for use in absorbent sanitary articles such as, for example, nappies for children and adults, to which express reference is hereinafter made without losing in generality, sanitary towels and the like.

### BACKGROUND ART

As is known, nappies comprise an absorbent pad or core which is normally enclosed between a permeable inner layer of non-woven fabric and an impermeable outer layer made, for example, of polyethylene.

Absorbent pads of known type comprise an absorbent core made of an absorbent material, such as, for example, granules of superabsorbent polymer material (SAP) inside a mixture of containment cellulose pulp (fluff) and absorbent material binder, sandwiched between two layers of non-woven fabric.

To improve performance in terms of absorption, comfort and distribution of absorbed liquids, pads have been developed which are provided with longitudinal channels without absorbent material between the two layers of non-woven fabric.

Document EP2905001 relates to an apparatus and a method for making an absorbent pad of this kind.

The apparatus described comprises rollers for transferring two sheets of non-woven fabric, a system for feeding the absorbent material and configured in such a way that the aforesaid channels are formed between the layers of non-woven fabric, and a system for applying adhesive used to join the two sheets.

According to the description, the rollers feed the sheets and the adhesive is spread on the sheets even at the channels; the absorbent material is spread discretely on one of the two sheets, trying to prevent it from being deposited on the channels.

For this purpose, the transfer roller on which the absorbent material is fed is provided with seats in which the pads are formed.

Each seat has suction zones where the absorbent material is held down and non-suction zones, including areas at the channels. One drawback of this system is due to the fact the non-suction zones create discontinuity in the suction which can cause the absorbent material to settle non-uniformly in the seat, or even to form lumps (especially at the suction zones).

These lumps may cause discomfort to the wearer of the nappy.

### DISCLOSURE OF THE INVENTION

In this context, the main purpose of this disclosure is to propose an apparatus for forming an absorbent pad to overcome the above mentioned disadvantages.

One aim of this disclosure is to propose an apparatus for forming an absorbent pad with channels which is more comfortable than prior art solutions.

Another aim of this disclosure is to propose an apparatus for forming an absorbent pad which allows making precise absorbent pads where the absorbent material is spread as evenly as possible.

These aims are fully achieved by an apparatus for forming an absorbent pad having the features resulting from the combination of one or more of the claims accompanying this application.

According to a first aspect of it, this disclosure relates to an apparatus for forming an absorbent pad for an absorbent sanitary article.

The pad comprises a first layer, a second layer and an absorbent material interposed between the first and the second layer and arranged according to a spreading pattern having at least one channel which is free of absorbent material.

The first and second layers are joined together at least along a first and a second longitudinal edge and along a first and a second transverse edge.

The forming apparatus comprises a forming drum, a first feed system for feeding to the forming drum a first web, intended to form the first layer, and a second feed system for feeding to the forming drum a second web, intended to form the second layer.

The forming apparatus comprises at least one spreader for spreading the absorbent material on the forming drum and the forming drum comprises a suction system to create, for example on the first web advancing on the forming drum, a spread of absorbent material according to the absorbent material spreading pattern.

According to one aspect of this disclosure, the forming drum comprises, in the suction system, at least one insert located at the pad channel to inhibit suction at the channel.

That way, the absorbent material is not retained at the insert, causing the channel to be formed.

The forming apparatus comprises at least one adhesive dispenser located upstream of the spreader of the absorbent material in a feed direction V of the first web to apply on the first web a layer of adhesive according to a gluing pattern and intended, for example, at least to retain the absorbent material and to contribute to joining the first and second webs.

Once joined to each other, the first web, the second web and the absorbent material form a composite web which is subsequently cut into pieces to obtain the aforesaid pads, each corresponding to a respective piece.

According to one aspect of the disclosure, the apparatus comprises a welding system for welding the first web to the second web to join the first web to the second web at least at the channel without absorbent material.

According to one aspect of this disclosure, the welding system comprises a first welding element and a second welding element acting in conjunction with each other.

The first welding element has an opposing surface for contact with the second welding element and shaped according to a welding pattern which comprises at least one welding zone at the channel.

The weld at the channel ensures a secure and reliable join.

According to one aspect of this disclosure, the welding pattern comprises a second welding zone at a first and a second longitudinal edge of the composite web so that the resulting pads have welded longitudinal edges which are more reliable than the glued edges of other solutions.

The two webs of non-woven fabric are joined by a permanent ultrasonic weld.

According to one aspect of this disclosure, the welding system is an ultrasonic welding system in which the first welding element is an anvil and the second welding element is a sonotrode acting on the anvil.

In one embodiment, the anvil is in the form of a welding roller comprising, on the outside surface of it, the opposing contact surface shaped according to the welding pattern.

According to one aspect of this disclosure, the welding roller, in one embodiment, is the forming drum which is thus provided with the opposing contact surface acted upon by the sonotrode.

The first welding element for example a roller which acts as anvil and provided with the welding pattern allows welding both the pad edges and the channels at the same time.

According to one aspect of this disclosure, the forming apparatus comprises a second welding system for welding the first web and the second web.

The second welding system comprises a third welding element and a fourth welding element acting in conjunction with each other.

The third welding element has a second opposing surface for contact with the fourth welding element and shaped according to a second welding pattern, different from the first welding pattern.

The second welding pattern comprises at least a second welding zone at the channel.

The first and second welding systems are used to weld absorbent pads of a first and second size, respectively, and operate alternatively; that makes the apparatus particularly versatile and capable of facilitating rapid changeovers to pads of different sizes.

The second welding system is preferably an ultrasonic welding system; when there are two welding systems, the welding system that is not in operation merely constitutes a transit for the composite web.

According to one aspect of this disclosure, the gluing pattern comprises a zone which is free of adhesive at the channel.

In one embodiment, the welding pattern comprises a third welding zone transverse to the second welding zone to define a transverse end of the pad. That way, when the composite web is cut into pieces, the pads will be welded at the transverse edges as well as at the channels.

According to one aspect of the disclosure, the forming apparatus comprises a calibrating system for calibrating the thickness of the absorbent material in the composite web.

The calibrating system is preferably located upstream of the welding system in the feed direction of the composite web.

In an embodiment, the calibrating system is located downstream of the second feed system which feeds the second web.

According to an aspect of the disclosure, the calibrating system, in an example embodiment, comprises a first roller having a first axis of rotation and a second roller having a second axis of rotation which is parallel to the first axis of rotation.

A first outside surface of the first roller and a second outside surface of the second roller delimit a slit for the passage of the composite web.

The width of the slit is fixed at least in a radial direction common to the first and the second roller.

According to an aspect of this disclosure, the width of the slit substantially corresponds to a preferred thickness of the absorbent pad.

According to an aspect of this disclosure, the width of the slit is less than a preferred thickness of the absorbent pad.

That way, when the composite web passes between the two rollers of the calibrating system, that is to say, through the slit, the absorbent material, specifically any lumps exceeding the predetermined thickness, is squeezed flat and spread more evenly within the pad so it will not cause discomfort to the wearer.

In a preferred embodiment, the outside surface of the first roller is a right-angled cylindrical surface having as axis the first axis of rotation.

In a preferred embodiment, the outside surface of the second roller is a right-angled cylindrical surface having as axis the second axis of rotation.

According to an aspect of the disclosure, the first and second rollers of the calibrating system extend in width along the respective axis of rotation at least as much as the composite web does so as to be able to squeeze the composite web along its full width.

According to one aspect of this disclosure, the apparatus comprises a removal system for removing absorbent material and operating at the forming drum.

The removal system for removing the absorbent material is located downstream of the absorbent material spreader in the feed direction V of the first web to remove any absorbent material that may be present on the first web at the channel.

Removing the material that may be present there allows a better quality weld to be made, thus obtaining a more reliable pad.

In one preferred embodiment, the removal system for removing the absorbent material comprises a blowing system comprising at least one centrifugal impeller whose axis of rotation is parallel to the axis of rotation of the forming drum.

The impellers preferably have an outside surface which faces the forming drum.

According to one aspect of the disclosure, the outside surface of the impellers is at least partly radially aligned with the inserts where the channels are formed.

According to one aspect of the disclosure, the impellers generate a vortex at the inserts which forces any absorbent material that may be present there towards negative pressure zones of the forming drum.

In such a case, the material blown away settles on the material already present on the suction zones and forms lumps which are then squeezed flat by the calibrating system.

In one embodiment, the removal system for removing the absorbent material may comprise a blow nozzle which is preferably directed at the forming drum in such a way as to blow away any absorbent material that may be present at the channel, on the first web.

According to an aspect of the disclosure, the forming apparatus comprises a pressing device for pressing the composite web along the first and second longitudinal edges of the pads.

The pressing device is located downstream of the calibrating system in the feed direction V of the composite web.

In a preferred embodiment, the pressing device is located upstream of the welding system of the first and second webs.

In a preferred embodiment, the pressing device comprises a third roller having a a third axis of rotation and an opposing member in contact with an outside surface of the third roller.

According to an aspect of the disclosure, the outside surface of the third roller has an annular groove delimiting a first and a second annular tooth which are axially spaced and intended to engage the composite web along the first and second longitudinal edges of the successive absorbent pads making up the composite web.

In use, the composite web passes between the third roller and the opposing member in such a way as to join the longitudinal edges of the pads together more effectively.

According to an aspect of the disclosure, the calibrating system, in one embodiment, comprises the joining device: that is to say, the same system performs both the function of calibrating and squeezing the absorbent material and that of pressing the longitudinal edges of the pads.

In a preferred embodiment, the opposing member is a fourth roller having a fourth axis of rotation parallel to the third axis of rotation and having a cylindrical outside surface defining with the outside surface of the third roller a passage for the composite web.

Preferably, therefore, according to an aspect of the disclosure, the composite web, in use, passes between the third and the fourth roller while the first and second annular teeth of the third roller press the composite web against the fourth roller along the longitudinal edges of the pads.

According to one aspect of it, this disclosure relates to a method for forming an absorbent pad for an absorbent sanitary article of the kind described above.

The method comprises a step of feeding a first web intended to form the first layer of the pad, a step of feeding a second web intended to form the second layer of the pad, a step of spreading the absorbent material on the first web according to the above mentioned absorbent material spreading pattern, and a step of spreading an adhesive on the first web according to a gluing pattern.

Once joined to each other, the first web, the second web and the absorbent material form a composite web.

According to an aspect of the disclosure, the method comprises a step of calibrating the thickness of the absorbent material in the composite web.

More specifically, the step of calibrating comprises a step of squeezing the composite web to give the composite web a uniform thickness.

Calibrating or squeezing the composite web, specifically the absorbent material therein, flattens out any lumps which may be present.

Preferably, the step of squeezing is carried out across the full width of the composite web.

According to an aspect of the disclosure, the step of calibrating, preferably accomplished by squeezing the composite web, comprises feeding the composite web through a slit of predetermined height which corresponding to the desired thickness of the composite web, or rather, of the absorbent material inside it.

Preferably, the height of the slit is less than the desired thickness of the composite web which is squeezed as it passes through the slit.

According to an aspect of the disclosure, the method comprises a step of pressing the composite web along the first and second longitudinal edges of the pads which, in practice, make it up.

Preferably, the step of pressing the composite web along the first and second longitudinal edges of the pads is carried out after the step of calibrating the thickness of the absorbent material in the composite web.

The step of pressing the longitudinal edges effectively restores the adhesive joint between the first and the second web along these edges, especially if the preceding step of calibrating has strained or weakened this adhesive joint.

According to an aspect of the disclosure, the method comprises a step of welding the first web and the second web, preferably ultrasonic welding, according to a welding pattern comprising at least one welding zone at the channel.

According to one aspect of this disclosure, the method comprises a step of removing any absorbent material that may be present in the welding zone at the channel.

The step of removing the absorbent material comprises a step of blowing away any absorbent material that may be present in the welding zone at the channel.

### BRIEF DESCRIPTION OF THE DRAWINGS

Further characteristics and advantages of this solution are more apparent in the non-limiting description below, with reference to a preferred but non-exclusive embodiment of a method and an apparatus for forming a pad, as illustrated in the accompanying drawings, in which:

FIG. 1 is a schematic top plan view of an absorbent pad made by an apparatus according to this disclosure;

FIG. 2 is a schematic front view of a forming apparatus according to this disclosure for forming an absorbent pad;

FIG. 3 illustrates the apparatus of FIG. 1 in a schematic side view, partly in blocks and with some parts cut away for greater clarity;

FIG. 4 shows a detail of the apparatus of FIG. 1 in a schematic side view and with some parts cut away for clarity;

FIG. 5 shows a detail of the apparatus of FIG. 1 in a schematic side view and with some parts cut away for clarity.

### DETAILED DESCRIPTION OF PREFERRED EMBODIMENTS OF THE INVENTION

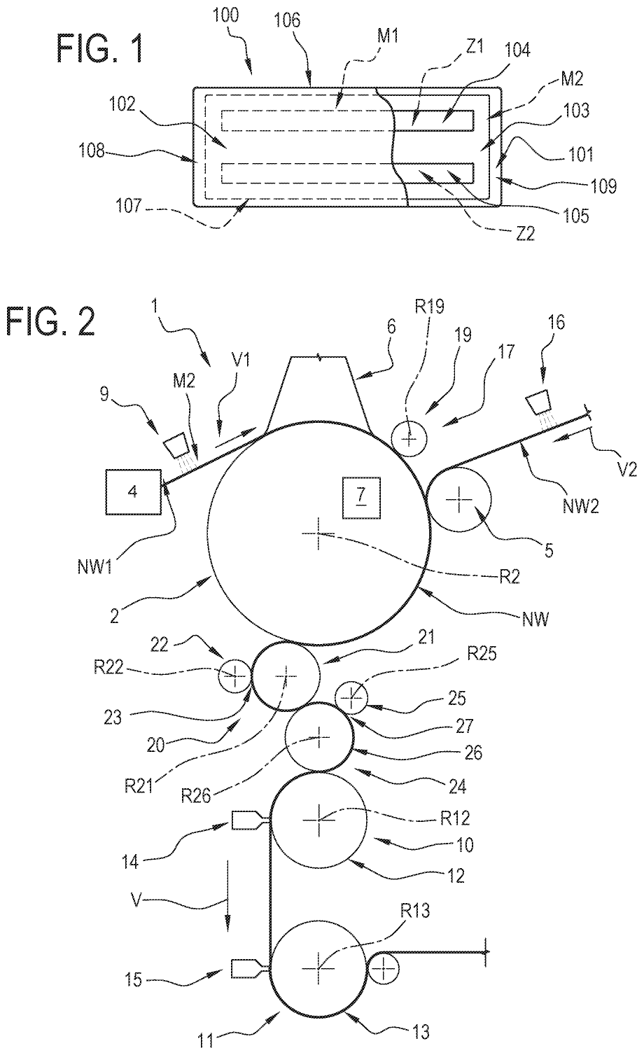

With reference to FIG. 1, the numeral 100 denotes a pad obtainable with a forming apparatus made in accordance with this disclosure.

The pad 100, intended for use in absorbent sanitary articles such as nappies for children or adults, for example, comprises a first layer 101, a second layer 102 and an absorbent material 103 interposed between the first and the second layer 101, 102 and arranged according to a spreading pattern M1.

The first and second layers 101, 102 are made, for example, of non-woven fabric and are joined to each other; the absorbent material 103 comprises, for example, cellulose fibres and superabsorbent material, also called SAP, and is fixed between the first and the second layer 101, 102.

In the example illustrated, the pattern M1, and hence the pad 100, has two zones or channels 104, 105 which are free of absorbent material and where the first and second layers 101, 102 are joined directly to each other.

In the preferred embodiment illustrated by way of example, the pad 100 has a first and a second longitudinal edge 106, 107 along which the first and second layers 101, 102 are joined directly to each other, for example by adhesive and/or by welding.

In the preferred embodiment illustrated by way of example, the pad 100 has a first and a second transverse edge 108, 109 along which the first and second layers 101, 102 are joined directly to each other, for example by adhesive and/or by welding.

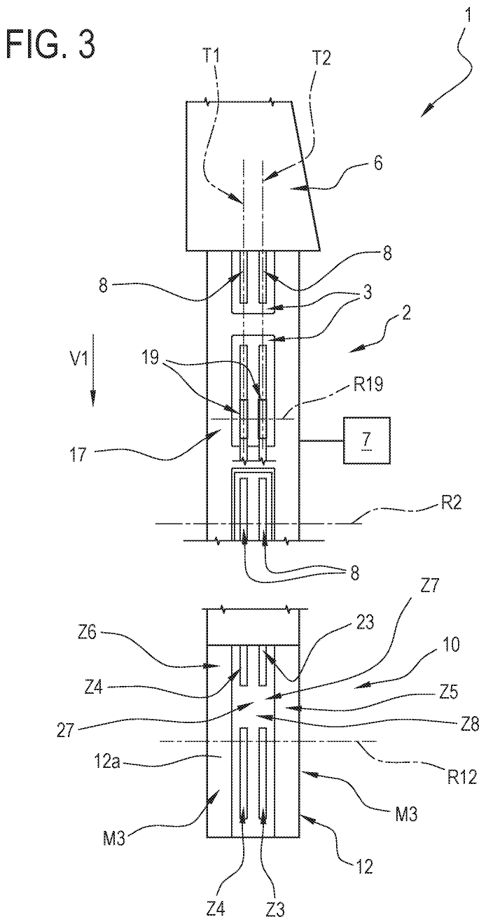

With reference to FIGS. 2 and 3, the numeral 1 denotes a forming apparatus for forming the pad 100 made in accordance with this disclosure.

This description of the apparatus 1 is limited to the parts necessary for understanding this disclosure.

The apparatus 1 comprises a drum 2 for forming the pads 100 and rotatable about an axis R2.

Along its periphery, the drum 2 comprises a plurality of seats 3, in particular for receiving the material 103, as described in more detail below.

The apparatus 1 comprises a first feed system, schematically represented as a block 4, for feeding a first web NW1 to the forming drum 2.

The web NW1 is movable in a direction V1 and is intended to form the first layer 101 of the pad 100.

The apparatus 1 comprises a second feed system, schematically represented as a block 5, for feeding a second web NW2 to the forming drum 2.

The web NW2 is movable in a direction V2 and is intended to form the second layer 102 of the pad 100.

The apparatus 1 comprises a spreader 6 for spreading the absorbent material 103 on the forming drum 2; the spreader 6 is of a substantially known type and is not described further.

The first web NW1, the second web NW2 and the absorbent material 103 together form a composite web NW which is movable in a direction V.

The forming drum 2 comprises a suction system 7, schematically represented as a block, to create on the first web NW1 fed by the drum 2, a spread of absorbent material according to the absorbent material spreading pattern M1.

More specifically, the suction system 7 is in communication with the seats 3 which, in practice, are preferably suction tiles and which hold the material 103 at a predetermined position relative to the web NW1, which is also held partly on the drum 2 by suction, according to the pattern M1.

The material 103 is positioned on the suction tiles which, in an embodiment not illustrated, may be in the form of a continuous circular seat extending along a substantially cylindrical peripheral portion of the drum 2.

In another embodiment, of the type illustrated in the drawings, the suction tiles may be in the form of a plurality of discrete seats 3, aligned and equispaced along a substantially cylindrical peripheral portion of the drum 2.

The seats 3 may in any case be shaped to match the pad 100 and are capable of retaining by suction the absorbent material 103 transported by the drum 2.

As may be observed in particular in FIG. 3, the forming drum 2 comprises a plurality of inserts 8 in the suction system 7, positioned in particular in the seats 3, to inhibit suction at the channels 104 and 105 to be formed in the pads 100.

That way, the absorbent material 103 is not held on the web NW1 where the inserts 8 are and hence, where the channels 104, 105 will be formed.

In the preferred embodiment illustrated, the forming apparatus comprises an adhesive dispenser 9 located upstream of the spreader 6 of the absorbent material in the feed direction V1 to apply on the first web NW1 a layer of adhesive according to a pattern M2 for gluing the absorbent material 103.

In a preferred embodiment, the gluing pattern M2 corresponds to the absorbent material pattern M1, that is to say, the adhesive is applied on the web NW1 in the zones where the absorbent material 103 is to be deposited.

More specifically, the gluing pattern M2 comprises zones Z1, Z2 which are free of adhesive, corresponding to the channels 104, 105.

A preferred embodiment of the apparatus 1 comprises a welding system 10 to join the first and second webs NW1, NW2 according to a welding pattern M3.

In the preferred embodiment illustrated by way of example, the apparatus 1 comprises a welding system 10 to join the first and second webs NW1 and NW2 according to a welding pattern M3 and a welding system 11 to join the first and second webs NW1 and NW2 according to a welding pattern M4.

The systems 10 and 11 are used to weld pads 100 of different sizes and operate alternatively, that is to say, when the system 10 is operating, the web NW passes through the system 11 in transit only, and vice versa.

Preferably, the apparatus 1 comprises an ultrasonic welding system 10, 11.

The systems 10 and 11 each comprise a first welding element 12, 13 which, in the example illustrated, is defined by a welding roller rotatable about a respective axis of rotation R12, R13.

The systems 10 and 11 each comprise a second welding element 14, 15 which, in the example illustrated, is defined by a sonotrode.

The rollers 12 and 13 each define, for the respective sonotrodes, a welding anvil.

Each first welding element 12, 13 has an opposing surface for contact with the respective second welding element and shaped according to the respective welding pattern M3, M4.

The welding roller 12 with the respective welding pattern M3 is illustrated by way of an example in FIG. 3; the pattern M4 is conceptually identical to the pattern M3 and is therefore not described further.

The welding roller 12 has an opposing surface 12a for contact with the sonotrode 14 and shaped according to the respective welding pattern M3.

The pattern M3 comprises a welding zone Z3, Z4 corresponding to the channels 104, 105.

In practice, the surface 12a is shaped to define an anvil for the sonotrode 14 in the zones Z3, Z4 corresponding to the channels 104 and 105.

In a preferred embodiment of the apparatus 1, the pattern M3 comprises a welding zone Z5, Z6 at the longitudinal edges 106, 107 of the pad 100.

In practice, the surface 12a is shaped to define an anvil for the sonotrode 14 in the zones Z5, Z6 corresponding to the longitudinal edges 106 and 107 of the pad 100.

In an embodiment not illustrated, the pattern M3 comprises a welding zone Z7, Z8 at the transverse edges 108, 109 of the pad 100.

In practice, the surface 12a is shaped to define an anvil for the sonotrode 14 in the zones Z7, Z8 corresponding to the transverse edges 108 and 109 of the pad 100.

In an embodiment not illustrated, the welding roller, that is, the first welding element of the welding system 10, is the forming roller 2.

In such a case, the forming roller 2, which in the case of ultrasonic welding, constitutes the anvil of the system, comprises a respective opposing surface for contact with the sonotrode and shaped, for example, according to the welding pattern M3.

In the embodiment illustrated by way of example, the apparatus 1 comprises a second adhesive dispenser 16 for applying a layer of adhesive on the web NW2 in order to optimize to the first web NW1.

The adhesive delivered by the dispenser 16 can cause the web NW2 to adhere to the web NW1, joining them together to form the composite web.

According to one aspect of this disclosure, the apparatus 1 comprises a removal system 17 for removing any absorbent material that may be present on the first web NW1 at the inserts 8, that is, in the zones of the web which will constitute the channels 104, 105.

The system 17 operates at the forming drum 2 and is mounted downstream of the spreader 6 of the absorbent material 103 in the feed direction V1 of the first web NW1.

In the embodiment illustrated by way of example, the removal system 17 for removing the absorbent material comprises a blowing system 18 which can blow away from the web NW1 any absorbent material 103 that may be present at the inserts 8, that is, at the channels 104 and 105. The material 103 may be blown onto the suction zones, where it is retained by the system 7.

In the embodiment illustrated, the blowing system 18 comprises at least one centrifugal impeller 19 whose axis of rotation R19 is parallel to the axis of rotation R2 of the drum 2.

In the example illustrated, the system comprises two coaxial impellers 19, each disposed at a respective channel 104, 105.

As illustrated schematically in FIG. 2, the impellers 19 have an outside surface which faces the drum 2.

According to one aspect of the disclosure, the impellers 19 generate a vortex at the inserts 8 which forces any absorbent material 103 that may be present there towards negative pressure zones of the drum 2.

In alternative embodiments, the blowing system comprises nozzles which blow air at the inserts 8; the nozzles are preferably directed towards the forming drum 2.

Preferably, the impellers 19, or the blowing nozzles, are disposed along the trajectories T1, T2 followed by the inserts 8 as the drum 2 rotates about the axis R2.

The impellers 19, or the blowing nozzles, are also optimized to prevent the blown air from modifying the pattern of the absorbent material 103 spread near the channels.

In alternative embodiments not illustrated, the removal system 17 for removing the absorbent material may, for example, comprise brushes acting on the web NW1 and/or a suction system for extracting the absorbent material and/or a scraper to ensure that the web NW1 remains clean at the channels 104, 105 where it will be welded.

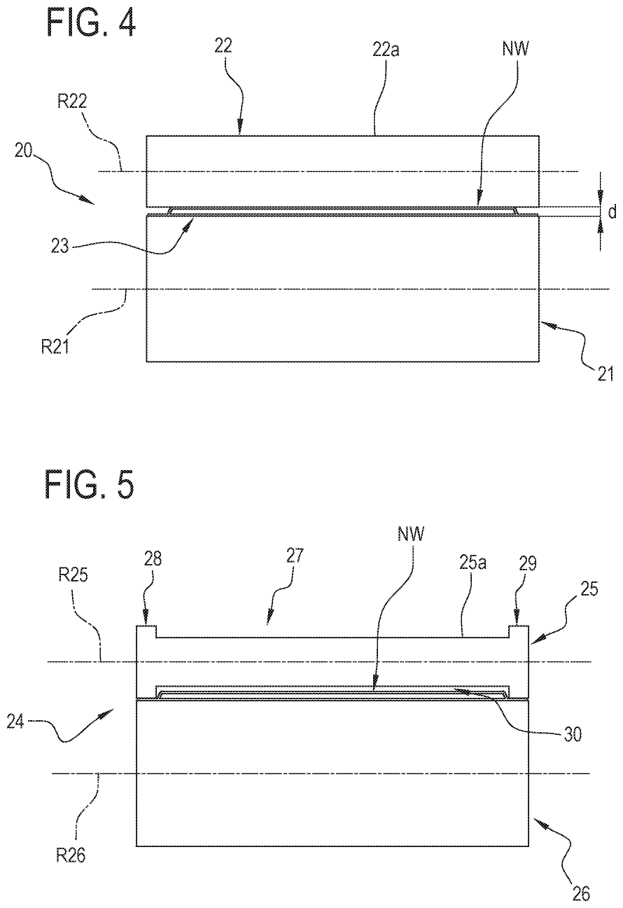

As may be observed in particular in FIGS. 2 and 4, the apparatus 1 in the preferred embodiment illustrated, comprises a calibrating system 20 for calibrating the thickness of the absorbent material in the composite web NW.

The calibrating system 20 is preferably located upstream of the welding system 10, 11 in the feed direction V of the composite web NW.

As illustrated, the calibrating system 20 is located downstream of the second feed system 5 which feeds the second web NW2.

The calibrating device illustrated by way of an example, comprises a first roller 21 having an axis of rotation R21 and a second roller 22 having an axis of rotation R22 which is parallel to the axis of rotation R21.

The axes R21 and R22 are preferably also parallel to the axis R2.

A first outside surface 21a of the first roller and a second outside surface 22a of the second roller delimit a slit 23 for the passage of the composite web NW.

The width “d” of the slit 23 is fixed at least in a radial direction common to the first and the second roller 21 and 22.

The width or height “d” of the slit 23 substantially corresponds to a preferred thickness of the absorbent pad 100.

In the preferred embodiment illustrated, the outside surface of the first roller 21 is a right-angled cylindrical surface having an axis R21 and the outside surface 22a of the second roller 22 is a right-angled cylindrical surface having an axis R22.

Preferably, the first and second rollers 21 and 22 of the calibrating system 20 extend in width along the axes R21 and R22 at least as much as the composite web NW does so as to be able to squeeze the composite web along its full width.

As may be observed in FIGS. 2 and 5, the apparatus 1 in the preferred embodiment illustrated, comprises a pressing device 24 for pressing the composite web NW along the first and second longitudinal edges 106, 107 of the pads 100.

The device 24 is located downstream of the calibrating system 20 in the feed direction V of the composite web NW.

In the preferred embodiment illustrated, the device 24 is located upstream of the welding system 10, 11 of the first and second webs NW1, NW2.

As illustrated by way of example, the device 24 comprises a third roller 25 having an axis of rotation R25 and an opposing member 26 in contact with an outside surface 25a of the roller 25.

The surface 25a preferably has an annular groove 27 delimiting a first and a second annular tooth 28, 29 which are axially spaced and intended to engage the composite web NW along the first and second longitudinal edges 106, 107 of the successive absorbent pads 100 making up the composite web NW.

In the preferred embodiment illustrated, the opposing member 26 is a fourth roller having an axis of rotation R26 parallel to the axis of rotation R25 and having a cylindrical outside surface 26a defining with the outside surface 25a of the third roller a passage 30 for the composite web.

In alternative embodiments not illustrated, the opposing member 26 may be a flat surface on which the web NW is fed.

In an alternative embodiment not illustrated, the calibrating system 20 comprises the pressing device 24 for pressing the composite web NW along the first and second longitudinal edges 106, 107 of the pad.

The pressing device 24 is located downstream of the feed system 5 for feeding the second web NW2 in the feed direction V of the composite web NW.

This alternative embodiment may be, for example, like the device illustrated in FIG. 5, where the depth of the groove 27, measured in the radial direction, corresponds to the width or height “d”.

A method for forming an absorbent pad 100 according to this disclosure comprises a step of feeding to the forming drum 2 the first web NW1, intended to form the first layer 101, a step of feeding to the forming drum 2 the second web NW2, intended to form the second layer 102, a step of spreading the absorbent material 103 on the first web NW1 according to the spreading pattern M1 and a step of spreading adhesive on the first web NW1 according to the gluing pattern M2.

The method comprises a step of joining together the webs NW1 and NW2, preferably by welding.

Welding the first and second webs NW1, NW2, for example ultrasonic welding, according to the welding pattern M3 or M4, which comprises the welding zones Z3, Z4 at the channels 104, 105, contributes to the formation of the composite web NW.

The method comprises a step of calibrating the thickness of the absorbent material 103 in the composite web NW.

Preferably, the step of calibrating comprises a step of squeezing the composite web NW to give the composite web NW a substantially uniform thickness or to evenly spread the absorbent material 103 inside it.

The step of squeezing, which is preferably carried out across the full width of the composite web NW, is accomplished by feeding the composite web through the slit 23.

This method comprises a step of pressing the composite web NW along the first and second longitudinal edges 106, 107 of the pads 100 which, in practice, make up the composite web NW.

In an embodiment, the step of pressing the composite web NW is carried out simultaneously with the step of calibrating the thickness of the absorbent material 103 in the composite web NW.

In practice, while the absorbent material is being squeezed to eliminate any lumps, the longitudinal edges of the pads are also squeezed simultaneously as the composite web is fed between a pair of suitably shaped rollers.

The method comprises a step of removing any absorbent material 103 that may be present on the web NW1 in the welding zones Z3, Z4 where the channels 104, 105 will be defined.

In a preferred embodiment, illustrated by way of example, the step of removing the absorbent material 103 comprises a step of blowing away from the web NW1 any absorbent material 103 that may be present in the welding zones Z3, Z4 at the channels 104, 105 in order to clean the web so it is ready for subsequent welding.

Since the channels 104, 105 are not continuous along the composite web NW, and hence the zones 104, 105 free of absorbent material on the web NW1 are not continuous, the step of blowing can be intermittent.

1. An apparatus for forming an absorbent pad for an absorbent sanitary article, the pad comprising a first layer, a second layer and an absorbent material interposed between the first and the second layer and arranged according to an absorbent material spreading pattern M1 having at least one channel (104, 105) which is free of absorbent material, the first and second layers being joined to each other along a first and a second longitudinal edge, along a first and a second transverse edge and at the channel,
the apparatus comprising
a forming drum,
a first feed system for feeding to the forming drum a first web, intended to form the first layer,
a second feed system for feeding to the forming drum a second web, intended to form the second layer,
at least one spreader for spreading the absorbent material on the forming drum, the forming drum comprising a suction system to create on the first web disposed on the forming drum a spread of absorbent material according to the absorbent material spreading pattern M1, the forming drum comprising at least one insert in the suction system to inhibit suction at the channel, the absorbent material not being retained at the insert,
the first web, the second web and the absorbent material together forming a composite web, the apparatus comprising a system for joining the first and second webs at least at the channel and located downstream of the second feed system, the apparatus being characterized in that it comprises
a system for calibrating the thickness of the absorbent material in the composite web, the calibrating system being located upstream of the joining system in a feed direction V of the composite web.

2. The apparatus according to claim 1, wherein the calibrating device comprises a first roller, having an axis of rotation R21, and a second roller, having an axis of rotation R22 parallel to the axis of rotation R21, a first outside surface of the first roller and a second outside surface of the second roller delimiting a through slit for the passage of the composite web, the slit having a size “d” which is fixed in a radial direction common to the first and second rollers and corresponding substantially to a preferred thickness of the pad.

3. The apparatus according to claim 2, wherein the first outside surface is a right-angled cylindrical surface whose axis is R21.

4. The apparatus according to claim 2, wherein the second outside surface is a right-angled cylindrical whose axis is R22.

5. The apparatus according claim 2, wherein the first and second rollers (21, 22) extend widthways along the axes R21 and R22 at least as much as the composite web does.

6. The apparatus according to claim 1, comprising a pressing device for pressing the composite web along the first and second longitudinal edges, the pressing device being located downstream of the second feed system in the feed direction V of the composite web.

7. The apparatus according to claim 6, wherein the pressing device is located downstream of the calibrating system in the feed direction V of the composite web.

8. The apparatus according to claim 6, wherein the pressing device is located upstream of the joining system.

9. The apparatus according to claim 6, wherein the pressing devices comprises a third roller having an axis of rotation R25 and an opposing member for contacting an outside surface of the third roller, the outside surface of the third roller having an annular grooved which delimits a first and a second annular tooth (28, 29) axially spaced from each other and intended to engage the composite web along the first and second longitudinal edges of the absorbent pads following each other consecutively in the composite web, the composite web, in use, passing in between the third roller and the opposing member.

10. The apparatus according to claim 9, wherein the opposing member is a fourth roller having an axis of rotation R26 parallel to the axis of rotation R25 and having a cylindrical outside surface defining with the outside surface of the third roller a passage for the composite web.

11. The apparatus according to claim 1, wherein the calibrating system comprises a pressing device for pressing the composite web along the first and second longitudinal edges, the pressing device being located downstream of the second feed system in the feed direction V of the composite web.

12. The apparatus according to claim 1, comprising a systems for removing any absorbent material there may be on the first web at the insert, the removal system being located downstream of the absorbent material spreader in the feed direction V1 of the first web, the system for removing the absorbent material being operative on the forming drum and comprising a blowing system for blowing away any absorbent material there may be on the first web at the insert.

13. A method for forming an absorbent pad for an absorbent sanitary article, the pad comprising a first layer, a second layer and an absorbent material interposed between the first and the second layer and arranged according to a spreading pattern M1 having at least one channel which is free of absorbent material, the method comprising
a step of feeding a first web, intended to form the first layer,
a step of feeding a second web, intended to form the second layer,
a step of spreading the absorbent material on the first web according to the spreading pattern M1,
a step of joining the first and second webs,
the first web, the second web and the absorbent material together forming a composite web, the method being characterized in that it comprises a step of calibrating the thickness of the absorbent material in the composite web.

14. The method according to claim 13, wherein the step of calibrating comprises a step of squeezing the composite web to give the composite web a uniform thickness.

15. The method according to claim 14, wherein the step of squeezing is carried out across the full width of the composite web.

16. The method according to claim 13, wherein the step of calibrating the composite web comprises feeding the composite web through a slit of predetermined height “d” which is less than or equal to the desired thickness of the composite web.

17. The method according to claim 13, comprising a step of pressing the composite web along the first and second longitudinal edges of the pad.

18. The method according to claim 17, wherein the step of pressing the composite web is carried out after the step of calibrating the thickness of the absorbent material in the composite web.

19. The method according to claim 17, wherein the step of pressing the composite web is carried out simultaneously with the step of calibrating the thickness of the absorbent material in the composite web.

20. The method according to claim 13, comprising a step of removing any absorbent material there may be on the first web at the channel, the step of removing comprising a step of blowing away any absorbent material there may be on the first web at the channel.

21. The method according to claim 20, wherein blowing is intermittent.
